(19) United States
(12) Patent Application Publication
(10) Pub. No.: US 2004/0230116 A1
(43) Pub. Date: Nov. 18, 2004

(54) Method and apparatus for detection of ultrasound transducer failure in catheter systems

(75) Inventors: Mark W. Cowan, Fremont, CA (US); Paul D. Corl, Palo Alto, CA (US); Byron J. Reynolds, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PHARMASONICS, INC., Sunnyvale, CA (US)

(21) Appl. No.: 10/436,966

(22) Filed: May 12, 2003

(51) Int. Cl. A61B 8/00

(52) U.S. Cl. 600/437

(57) ABSTRACT

Methods and systems for detecting ultrasound transducer failure in an ultrasound catheter system comprise providing a memory device or other data storage element or catheter body having at least one ultrasound transducer disposed. The memory device stores a test current amplitude value which relates to an actual operating peak current for the at least one ultrasound transducer. An average actual operating peak current amplitude during a first period of time is calculated, and an actual operating peak current for the at least one ultrasound transducer over a second period of time may optionally also be calculated. Transducer failure has occurred if the actual operating peak current amplitude passes outside of a fit preferred range during the firs period of time, or the actual operating peak current amplitude passes outside of a second preferred range during a second period of time.

### BACKGROUND OF THE INVENTION

The present invention relates to catheter-based therapeutic ultrasound systems in general and to systems for detecting ultrasound transducer failure in catheter systems in particular.

Catheter systems employing high-output therapeutic ultrasound transducers can fail during operation for a variety of reasons, including the presence or development of microscopic fractures or other defects in the ceramic of the transducer. Subsequent to the development of such fractures, at the high drive energies typically applied, numerous harmonic vibration modes may develop among the now-independently vibrating elements of the transducer. As a result, much of the drive energy delivered to the transducer is converted into heat, not into acoustic energy as desired. Heat results from frictional losses between the microscopic regions of the ceramic. Such unintended heating not only reduces the generation of ultrasonic energy, but if sufficient heat is transferred to surrounding tissue it may also cause serious complications such as bums, or if applied within the vasculature, thrombus formation and burning of the arterial wall. Thus, a strong need exists to be able to detect transducer failure in high-output therapeutic ultrasound transducers.

A variety of approaches currently exist for detecting transducer failure in ultrasound systems, each suffering from various limitations when applied to catheter-based systems, as follows. First, a temperature-sensing device, such as a thermocouple or thermistor, can be attached directly to the ultrasonic transducer to provide feedback of transducer temperature. In such systems, the temperature-sensing device may be incorporated into a circuit that produces a voltage proportional to the temperature of the sensor. When this voltage exceeds a pre-set maximum value, a separate circuit detects this condition and either reduces the amplitude of the drive signal to the transducer or inhibits its operation entirely.

This first approach has numerous limitations, particularly when applied within an intravascular ultrasound catheter. The inclusion of a temperature measurement device and subsequent electrical wiring and connections into the catheter increases both manufacturing complexity and cost. The inclusion of such components further requires a significant amount of space, regardless of the state of their own miniaturization. In the case where a plurality of transducers are used, an independent temperature sensor is required for each transducer, further adding to the overall cost/complexity of the system. Additionally, the inclusion of a temperature sensing device adjacent each transducer element would not necessarily detect local overheating should a transducer fail at a point opposite the location of the sensor.

In a second approach, transducer impedance is monitored to detect a change in impedance away from desired operating characteristics. Unfortunately, in many instances, transducer failure may not result in a measurable change of impedance. For example, at high drive levels used during normal operation, slight changes in the phase between the driving voltage and current may be observed, but detection of the resulting impedance change may not be reliable, in particular when only one of multiple transducers has failed.

In a third approach, an ultrasonic receiving element (which may either be separate from, or fabricated as part of, the transmitting element) is used to detect a reduction in acoustic emissions, and thus is used to detect transducer failure. This third approach, however, suffers from significant cost and complexity in the form additional of receiving and detection circuitry to process the returned ultrasound signal. Moreover, the inclusion of such components requires a significant amount of space, regardless of the state of their own miniaturization. Furthermore, in the case where a plurality of transducers are used, an independent ultrasonic receiving element would be required for each transducer, further adding to the overall cost and complexity of the system.

In a fourth approach, non-invasive systems for the detection of temperature increases within the body are used. Such systems may include techniques adopted primarily for the localization and control of hyperthermia treatment of tumors. However, since such systems typically involve temperature estimation through the analysis of data obtained from non-invasive ultrasound imaging, MRI, or CT imaging, they are all costly and generally incompatible with the cardiac or peripheral catheterization laboratory setting.

For these reasons, it would be desirable to provide additional and improved methods and systems for detecting transducer failure during use of high-output therapeutic ultrasound devices, particularly those used in intravascular and other interventional procedures. Such methods and systems should require minimum modification of and/or addition to the design of and fabrication procedures for existing ultrasonic transducers and catheter-based transducer systems, should be capable of reliably detecting transducer failure, should be relative simple and inexpensive to implement, and should require very little extra space or weight in the deployed system. At least some of these objectives will be met by the inventions described hereinafter.

### BRIEF SUMMARY OF THE INVENTION

The present invention provides improved methods, systems, and apparatus for detecting transducer failure in medical ultrasound systems, particularly high-output therapeutic ultrasound systems where the transducer is remotely located in the vasculature or other body locations. The present invention is based at least partly on the recognition that power consumption in a properly operating ultrasonic transducer is very predictable, particularly in high-output transducers as defined below. Thus, any deviations in observed power consumption, particularly current consumption, from a predicted or calculated consumption pattern will provide an indication that the transducer has failed or is at significant risk of failure. In such cases, a signal or alert may be provided to the user and/or the system may be automatically shut down.

The present invention is particularly suitable for use with high-output therapeutic intravascular ultrasound systems such as those described in U.S. Pat. Nos. 5,725,494; 5,728,062; 5,735,811; 6,221,038; 5,846,218; 6,287,272; 6,464,660; 5,931,805; 6,228,046; 6,210,393; 6,372,498; 6,296,619; and 6,464,680, each of which is assigned to the assignee of the present application and each of which is incorporated herein by reference. By high-output transducer, it is meant that the transducer (or the combined transducers in multiple transducer systems) will have a peak acoustic output of at least about 1000 watts, usually at least about 100 watts. Typically, the peak acoustic output will correspond to a peak current consumption of at least about 5 amps, usually at least about 20 amps. The methods, systems, and apparatus, of the present invention are not limited to such high-output systems, but will find their greatest use with such systems.

In a first aspect of the present invention, methods for detecting failure of an ultrasound transducer in a remotely positioned therapeutic device comprise determining a first operational range, typically a power or current range, for the transducer based on data stored in or on the device. Operation of the transducer is then monitored by observing the operation over a first time period, and a failure is detected if the transducer operates outside of the determined first operational range during at least a predetermined portion of the first time period. As described in more detail below, the data stored in or on the device will preferably be obtained by testing the actual device which is incorporated into the catheter or other system. By testing the actual device, an operational characteristic of that particular device (as opposed to that class of device or devices fabricated in the same way as the particular device) can be precisely determined. By then observing the actual operation of the device, typically by measuring power or current consumption, deviations from the expected operational characteristics of the particular device being used may then be assessed.

Preferably, this method for detecting failure of the ultrasound transducer additionally includes a second phase or stage. By observing the operation of the transducer during the first time period, the actual operating characteristics of the device at the time during which it is being used may be determined. That is, any variations in the expected operation based on aging of the transducer, environmental conditions, or the like, may be taken into account, and a second expected operational range may then be calculated. Typically, the second operational range will be narrower than the first operational range so that operation during a second time period, which is usually continuous with the first time period, may then be assessed more rigorously. Typically, the second operational range will be based on the observed average cordial power or current consumption during the first time period. The range is then determined by placing upper and lower limits around the observed average, typically being 95% to 105% of the average, although such “window” may vary depending on the device and other factors. The operation of the transducer may then be assessed during such second time period, which may last as long as the entire remainder of the operation of the device, and any variations or deviations outside of the operational range may indicate a transducer or system failure. Particular limits or constraints on the deviations may vary so that some minimum time of operation outside of the second operational range may be required before a system failure is indicated.

In further preferred aspects of this first method, the data stored in or on the device is an actually measured current or power consumption, and the first operational range is calculated relative to this first value. Because of the operational characteristics of the transducer, the first operational range in some instances may actually be higher than the measured current or power consumption. For example, the first operational range may have a current value which is from 110% to 130% of the measured or test current value which is provided on the catheter itself.

The duration of the first time period will typically be relatively short, usually being from five seconds to thirty seconds, more typically being about 15 seconds measured from the time the transducer is initially energized. Failure of the transducer during the first time period may be indicated or detected when the transducer operates outside of the first operational range for some minimum time period ranging from a fraction of a second to a few seconds, typically being from about one second to five seconds.

The therapeutic ultrasound system will often operate in a series of bursts where the system is alternately energized and de-energized. During each burst, the transducer will be energized over a plurality of current waveform cycles. In such instances, observing the operation of the transducers will typically comprise measuring the peak current value after the first several current waveform cycles have passed, typically after at least four current waveform cycles have passed.

In a second aspect of the present invention, failure of an ultrasound transducer in a remotely positioned therapeutic device may be detected without the need to store or obtain power or current consumption characteristics of the particular device which is being used. Instead, such methods rely on measuring peak cycle current during operation of the device, typically by monitoring the current delivered to the device from an ultrasound generator. The difference between the peak current measured at an early cycle and the peak current measured at a subsequent or later cycle is then calculated. It has been found that the peak current value at an early cycle, typically the second or third cycle, and more typically the second cycle, should be higher than that observed at subsequent cycles, typically the fifth cycle or a later cycle. Thus, if the calculated difference between the measured cycle falls below an expected minimum value, typically at least 25% of the early peak current cycle value, then failure of the transducer is indicated.

In a further aspect of the present invention, a therapeutic ultrasound controller for use in combination with a catheter having a high-output therapeutic ultrasound transducer comprises means for measuring peak current delivered to the transducer. The means for determining a first expected peak current operational range for the transducer is further provided, typically based on a value received from an electronic memory in the catheter itself. Means for comparing the measured peak current value with the first determined peak current range is further provided, or measured peak current value which falls outside of the first determined peak current range indicates transducer failure. The system will usually further comprise means for determining a second expected peak current operational range based on the peak current measured during the first operational period, typically on the average peak current during the first operational period. Means are then provided for comparing the measured peak current value with the second determined peak current range, wherein a measured peak current value which falls outside of the second peak current range indicates a transducer failure.

In a fourth aspect of the present invention, a therapeutic ultrasound controller for use in combination with a catheter having a high-output therapeutic ultrasound transducer comprises means for measuring peak current to the transducer and means for comparing the measured peak current of an early current cycle with that of a later cycle, typically being the difference between the second or third peak current cycle with the fifth or later peak current cycle. Usually, the peak current cycles will be monitored in each burst of the transducer operation, where a difference of less than 25% between the peak current value indicates a failure of the transducers.

In yet another aspect of the present invention, an intravascular catheter comprises a catheter body, a high-output therapeutic ultrasound transducer operatively disposed on the catheter body, and data on or in the catheter body representing a measured operational range of the transducer. The data may be incorporated on the catheter body itself, on an attached hub, on an attached electrical connector, or on any other component or system of the catheter. A high-output therapeutic ultrasound transducer will typically have a peak acoustic power output of at least about 1000 watts, usually at least about 100 watts. The data may be incorporated in an electronic memory device which is in or on any portion of the catheter body, catheter hub, transducer assembly, electrical connector, or the like. Suitable electronic memory devices include flash memory, electrically-erasable memory (EEPROM), radio frequency identification tags (RFID's), and the like. Alternatively, the data may comprise indicia printed or otherwise embossed on to the catheter body or any related portion thereof. Indicia may be machine readable, e.g., being in the form of barcode, or may be human readable. In the latter case, an operator reading the operational data of the catheter can manually input such data into the catheter power supply or controller related to the power supply for practicing the methods of the present invention.

In a still further aspect of the present invention, methods for fabricating intravascular catheters comprise providing an intravascular catheter having a catheter body and a high-output therapeutic ultrasound transducer operatively disposed on or in the catheter body. A power consumption characteristic of the transducer during operation is then measured during or after the time of fabrication. Information representing the power consumption is then embedded in data in or on the catheter body in a readable form. Usually, measuring will comprise measuring current consumption of the transducer, typically where the current is measured with the transducer immersed in water and under nominal excitation conditions. Embedding may then comprise storing the data in an electronic memory disposed in or on the catheter body. Alternatively, embedding may comprise printing indicia setting for the measured value, where the indicia may be machine readable, e.g., in the form of barcode, or may be human readable. As described previously, the data encoded may represent any value which may be related to an expected operational range of the transducer based on the measured power consumption characteristic. For example, the data may be a peak power or current consumption value where the expected operational range is then calculated from the value. Alternatively, the data may represent the expected range itself, multiple expected ranges, etc.

### BRIEF DESCRIPTION OF THE DRAWINGS

FIG. 1 is an illustration of peak operating current of an ultrasound catheter over an extended period of time.

FIG. 2 is an enlarged view of a small portion of the instantaneous and peak operating currents of the catheter of FIG. 1, also showing the excitation voltage applied to the transducer to generate this current.

FIG. 3A is a system for generating the excitation voltage to, and detecting and monitoring peak current in an ultrasound transducer in a catheter system.

FIG. 3B is an alternate system for generating the excitation voltage to, and detecting and monitoring peak current in an ultrasound transducer in a catheter system.

FIG. 3C is another alternate system for generating the excitation voltage to, and detecting and monitoring peak current in an ultrasound transducer in a catheter system.

FIG. 4 is a flowchart of the operations of the present transducer failure detection system.

FIG. 5 is an illustration of both instantaneous and peak current waveforms generated by a properly functioning ultrasound transducer, corresponding to FIG. 2.

FIG. 6 is an illustration of both instantaneous and peak current waveforms generated by a degraded transducer.

FIG. 7 illustrates a system comprising a high-output therapeutic ultrasound catheter in combination with a therapeutic ultrasound controller in accordance with the principles of the present invention.

FIG. 8 illustrates a first configuration of the hub of the catheter of FIG. 7, shown with an electronic memory module incorporated in a side branch of said hub.

FIG. 9 illustrates a second configuration of the hub of the catheter of FIG. 7 shown with a barcode identifying the transducer characteristics of the catheter.

FIG. 10 illustrates a configuration of the electrical connector of the catheter of FIG. 7 shown with an electronic memory module incorporated within the body of said connector.

### DETAILED DESCRIPTION OF THE INVENTION

The present invention sets forth a method and system that can reliably and quickly detect the degradation or failure of an ultrasound transducer in a catheter system, such as a catheter system adapted to deliver therapeutic ultrasound energy to a patient. Advantageously, the present invention can also detect the degradation or failure of a single ultrasound transducer in a catheter system having a plurality of ultrasound transducer elements.

As will be explained, the present invention detects transducer degradation or failure by measuring the peak current through the transducer(s) over time. Deviation of this measured operating current outside of preferred current ranges during particular periods of time (and for particular lengths of time) results in a failure detection condition. As will be explained, these preferred current ranges correspond to pre-stored “test” characteristics that are particular to the individual catheter in use.

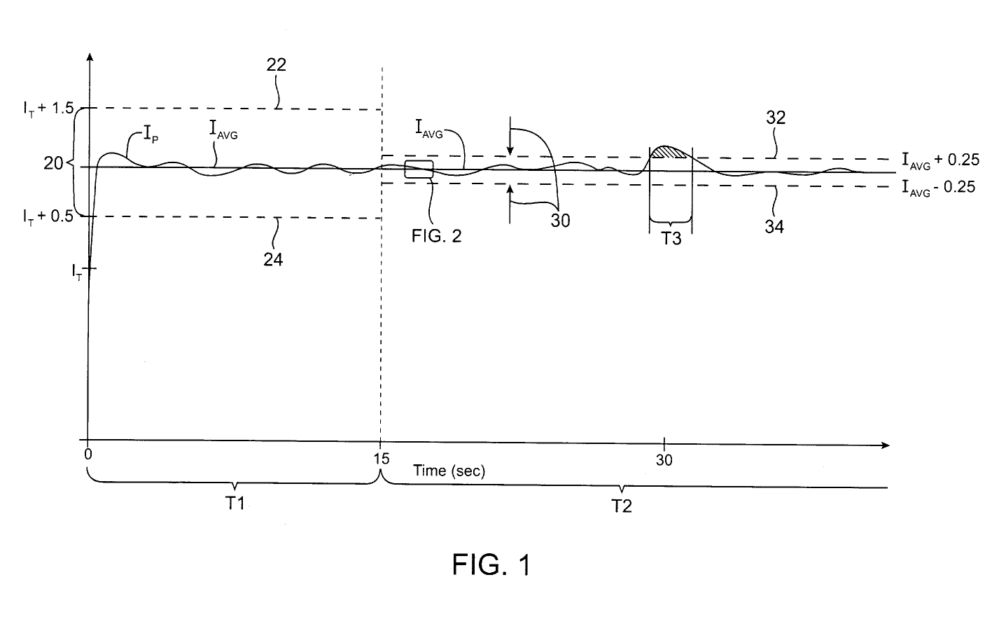

FIG. 1 is an illustration of peak operating current (I_{P}) of an ultrasound catheter as measured over an extended period of time.

In accordance with the present invention, a memory device is provided and is used to store a “test” current value (I_{T}) therein. Preferably, this memory device is provided within the body or hub or electrical connector of the catheter, as will be explained. Test current value I_{T} preferably corresponds to an experimentally determined (or expected) peak operating current value for the particular catheter in use. This I_{T} value is preferably determined during the manufacture of the particular catheter in use. As such, each catheter may have its own (i.e., different) I_{T} current value stored therein. In preferred aspects, this peak operating current I_{T} is typically between 4.0 and 8.0 A when measured in room temperature water for a catheter incorporating a multi-element transducer.

It has also been experimentally determined by the present inventors that peak operating current for such an ultrasound catheter is typically higher when measured in-vivo (due to changes in transducer impedance from temperature and acoustic loading). For example, in-vivo peak current typically ranges from 0.5 A to 1.5 A higher than the peak current observed with the transducers in water. Accordingly, the actual measured peak operating current I_{P} is thus typically between 0.5 A and 1.5 A above the stored test current I_{T}.

In accordance with the present invention, therefore, the present system detects whether actual peak operating current I_{P} is within a first range 20 during a first period of time T1 (i.e., transducer failure is determined to have occurred if I_{P} exceeds upper limit 22 or falls below lower limit 24 during time T1). Should I_{P} exceed upper limit 22, this would indicate failure due to transducer fracture or degradation, or an electrical short circuit. Conversely, should I_{P} fall below lower limit 24, this would indicate failure due to a transducer or an electrical connection which has been broken by excessive bending or mishandling of the catheter prior to use.

In preferred aspects, therefore, upper limit 22 is preferably equal to I_{T} plus approximately 1.5 A; and lower limit 24 is preferably equal to I_{T} plus approximately 0.5 A.

In various preferred aspects, lower limit 24 is preferably approximately equal to 110% of I_{T} and upper limit 22 is preferably approximately equal to 130% of I_{T}.

Transducer degradation during operation due to microscopic breakdown of the ceramic causes a rise in steady-state peak current, although this increase may be subtle, especially in the case of breakdown of a single transducer element in a multi-element catheter. In such a case, simply monitoring peak current against fixed limits such as would be suggested by the above described monitoring in first range 20, i.e., between upper limit 22 (I_{T}+1.5 A) and lower limit 24 (I_{T}+0.5 A) may not be sensitive enough to detect this transducer degradation. For example, in the case of a multi-element transducer operating at, or near lower limit 24 of this range (i.e., operating at or near I_{T}+0.5 A) which suffers a single element breakdown, the measured current I_{P} may rise significantly, but still remain within the ‘normal’ range (i.e., below upper limit 22).

It has also been experimentally determined by the present inventors, that although the in-vivo peak current I_{P} may be highly variable with respect to in-vitro current levels, it does remain quite stable during normal operation in a given in-vivo setting, typically varying less than +/−5% (i.e., about +/−0.25 amperes) from an initial I_{P} value.

In accordance with the present system, therefore, a determination is also made whether the peak current I_{P} has increased or decreased by more than about 5% or 0.25 A from the average of the peak operating current I_{P} measured during time period T1, after time period T1 has passed. In preferred aspects, T1 is approximately 15 seconds in duration.

During time period T1, an average peak current intensity I_{AVG} (being the average of I_{P}) is determined. After time T1, (i.e., any time after the start of time period T2), the present system detects whether actual peak operating current I_{P} remains within a second range 30. Range 30 is preferably centered at the value determined for I_{AVG}. In accordance with preferred aspects, range 30 has an upper limit 32 and a lower limit 34, as shown. In one preferred aspect, transducer failure is determined to have occurred if I_{P} exceeds upper limit 32 or falls below lower limit 34 during time T2.

In one preferred aspect, upper limit 32 will be about 5% or 0.25 A greater than I_{AVG}, and lower limit 34 will be 0.25 A lower than I_{AVG} As can be seen, therefore, range 30 will be much narrower than range 20. As such, a smaller variance in I_{P} during time T2 will result in a failure detection condition. An advantage of range 30 being narrower than range 20 is that should only one of a plurality of transducers fail, the overall operating current for the multi-transducer system would only increase by a small amount. Such a small increase would nevertheless result in I_{P} exceeding upper limit 32.

An advantage of detecting transducer failure during time period T2 is that the present system is essentially comparing its measured current I_{P} to its own initial operating conditions. As such, failure detection during time period T2 (i.e., with I_{P} rising above upper limit 32 or falling below lower limit 34) results in detection limits which are particular to the individual catheter in use and the in-vivo conditions to which it is exposed.

In an optional preferred aspect of the invention, small deviations of I_{P} outside of range 30 are permitted, provided however, that I_{P} returns to a value within range 30 within a short period of time T3. In preferred aspects, T3 is limited to a pre-determined duration of less than 15 seconds. This optional “temporal filter” feature of the invention is advantageous, as follows.

Certain events, such as the injection of x-ray contrast media into the artery, can cause significant changes in the acoustic environment that temporarily affects the impedance, and thus the peak current, of the transducer. Accordingly, if I_{P} remains outside of range 30 for longer than a desired period of time, a conclusion can be made that one or more of the transducers are defective, and the system can then be shut down. Conversely, allowing I_{P} to briefly exit range 30 permits short duration events (such as the injection of x-ray contrast media into the artery) without triggering a transducer “failure detection” condition.

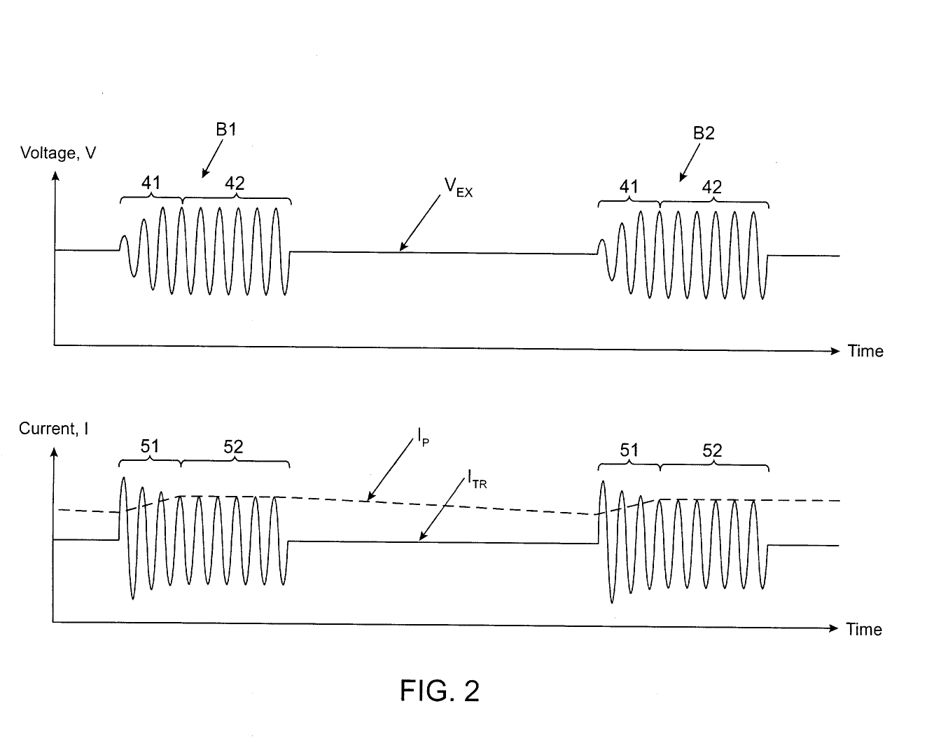

Referring next to FIG. 2, an enlarged (i.e., close up) view of the waveforms giving rise to a small portion of the peak operating current I_{P} of the catheter of FIG. 1 is shown. The waveform shown in FIG. 2 is exemplary of peak operating current I_{P} and the underlying excitation voltage waveforms of I_{P} at all times (i.e., during any of time periods T1, T2 or T3).

As can be seen in the enlarged view of FIG. 2, the line representing peak operating current I_{P} on FIG. 1 is actually comprised of the detected peak values of the instantaneous transducer current (I_{TR}), from a series of repeating excitation bursts (for example, B1 and B2). FIG. 2 also shows the excitation voltage applied (V_{EX}) to the catheter resulting in the instantaneous transducer current (I_{TR}) and detected peak current (I_{P}). V_{EX} and I_{TR} are shown contemporaneously. In other words, waveform cycles 51 of I_{TR} occur when waveform cycles 41 of V_{EX} occur; and, waveform cycles 52 of I_{TR} occur when waveform cycles 42 of V_{EX} occur, as follows.

It is well known that an ultrasound transducer can be modeled as a resonant circuit having a certain quality factor Q, with the value of Q being dependent on many variables in the design and construction of the transducer. As can be seen in FIG. 2, for each excitation burst (B1 or B2), several initial waveform cycles of V_{EX} are required prior to generating a steady-state condition in the transducers, this number of cycles being approximately equal to the Q of the resonant circuit, which for example in FIG. 2 is approximately 4. Specifically, for each excitation burst B1 or B2, the instantaneous transducer current I_{TR} is demonstrated by initial waveforms 51 of decreasing amplitude, prior to achieving steady-state waveforms 52.

In accordance with an optional preferred aspect of the present invention, therefore, I_{P} is therefore preferably calculated after 4 or even 5 waveform cycles of each excitation burst. (i.e., I_{P} is calculated only during waveforms 52). Accordingly, degradation of the described transducers during operation is preferably detected by only measuring the peak current of the fourth or subsequent cycles of each excitation burst.

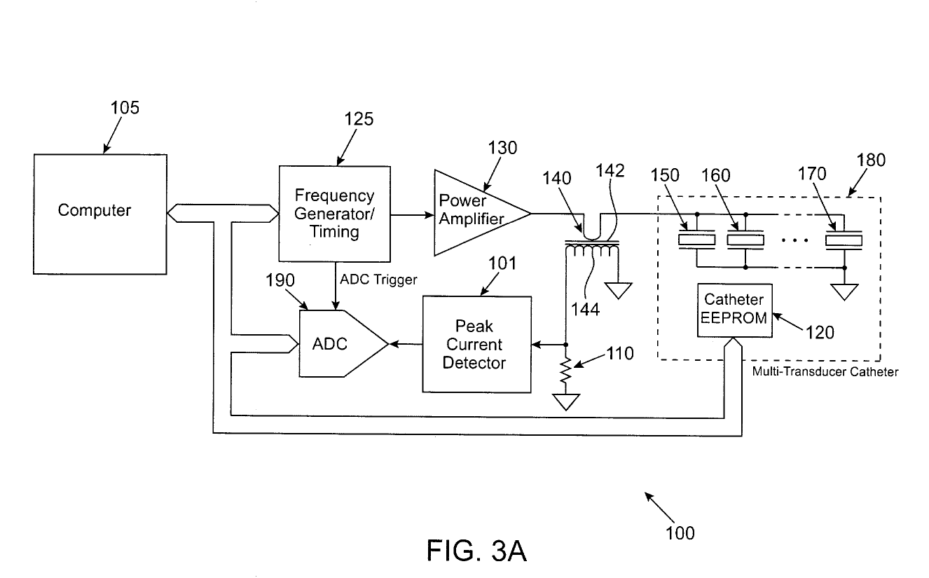

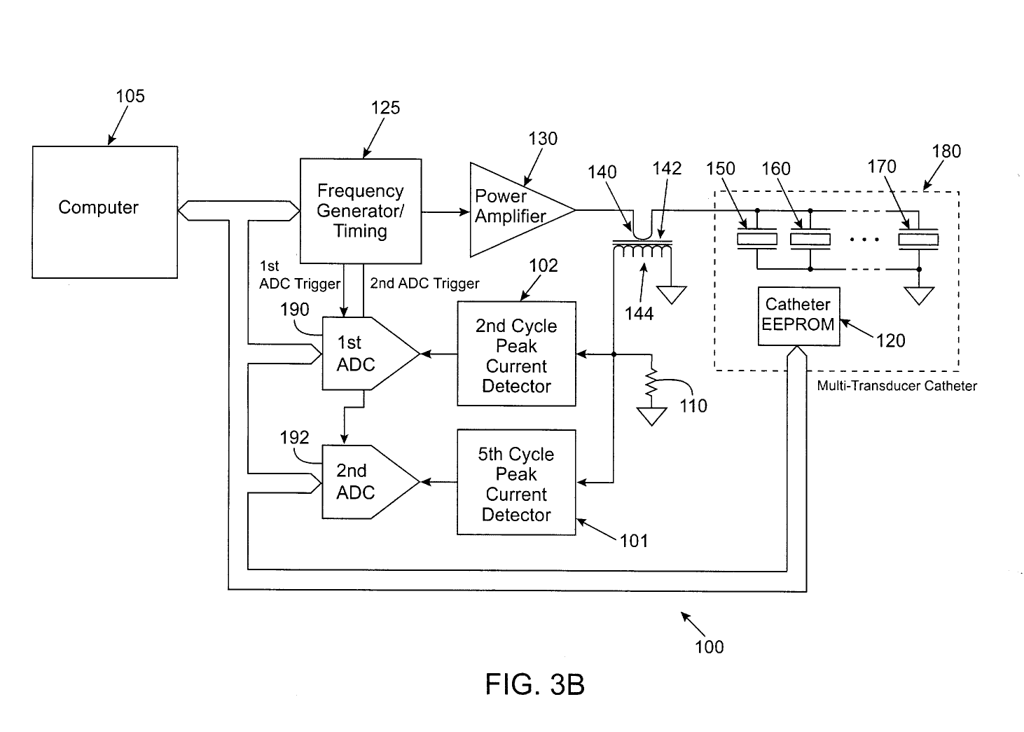

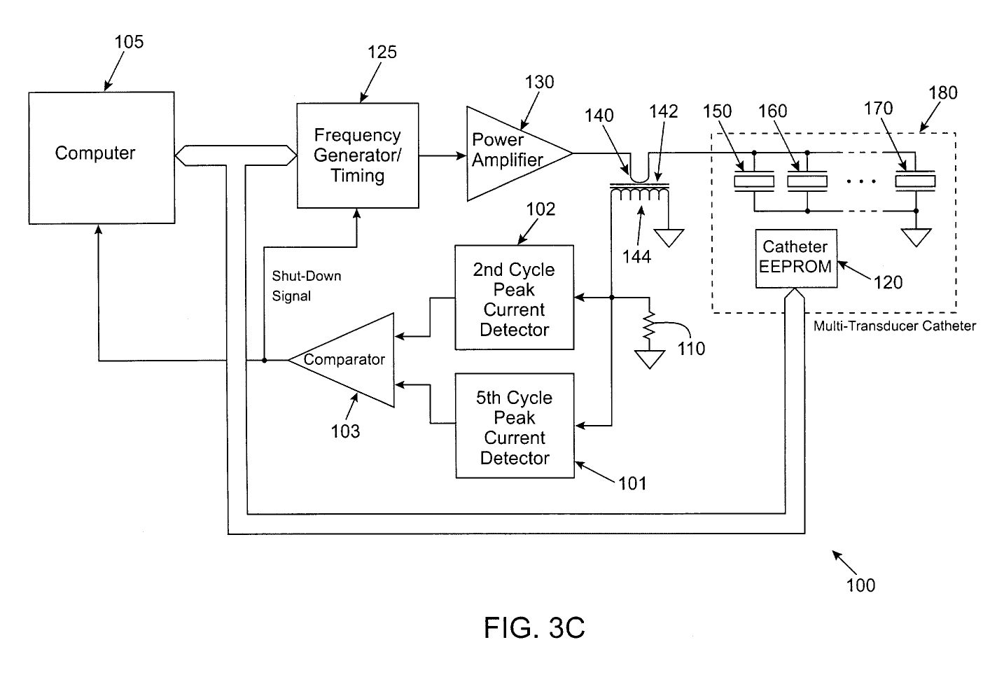

Referring next to FIG. 3A, a system 100 is provided for accomplishing the above method. A catheter 180 that may comprise a plurality of transducers 150, 160 and 170 is provided. Catheter 180 has an internal memory device 120 (which may optionally comprise an EEPROM) incorporated therein. Memory device 120 preferably stores information that may include test current (I_{T}) data recorded during manufacture of the catheter. As stated above, test current I_{T} preferably comprises the steady-state peak current amplitude that was experimentally determined (in water) for the particular catheter 180 being used.

System 100 also preferably comprises a current-detecting transformer 140 that produces a current in its secondary winding 144 proportional to the current in its primary winding 142. Primary winding 142 is connected in series between the ultrasound power amplifier 130 and transducers 150, 160 and 170 (in catheter 180). Therefore, the current passing from current-detecting transformer 140 through load resistor 110 creates a voltage signal that is also proportional to the transducer current. In a preferred aspect, the voltage across load resistor 110 is scaled and peak-detected by detection circuit 101 which, in a preferred aspect, has a peak acquisition delay equal to approximately four or five periods of the transmit waveform, corresponding, as stated above, to the number of waveform cycles required to achieve steady-state operation. In various preferred aspects, detection circuit 101 is preferably adapted to produce a voltage proportional to the peak current with an accuracy of better than +/−1% for peak currents ranging from 2.5 to 20 amperes.

The peak-detected current signal is then preferably digitized by an analog-to-digital converter 190, and the result is read by computer 105 for processing. Preferably, processing of the peak current signal in computer 105 includes reading test current I_{T} data (which may be recorded during manufacture of catheter 180, and stored in an EPROM memory device 120 incorporated into catheter 180), and processing test current I_{T} data in accordance with the presently set forth method, as described herein.

System 100 optionally comprises separate frequency generation and timing hardware 125, also preferably under control of computer 105, which preferably generates the signal to be amplified by power amplifier 130 and also optionally provides a timing signal to analog-to-digital converter 190 to initiate its sampling/data conversion cycle immediately following each transmit burst (e.g., burst B1). This timing offers the advantage of avoiding sampling the peak-detected signal either during the transmit cycle such as B1 (which could result in erroneous information due to internal electrical noise), or significantly after the transmit cycle has ended such as immediately before transmit burst B2 (which would result in under-reading the peak current due to unavoidable droop in the peak detection circuitry).

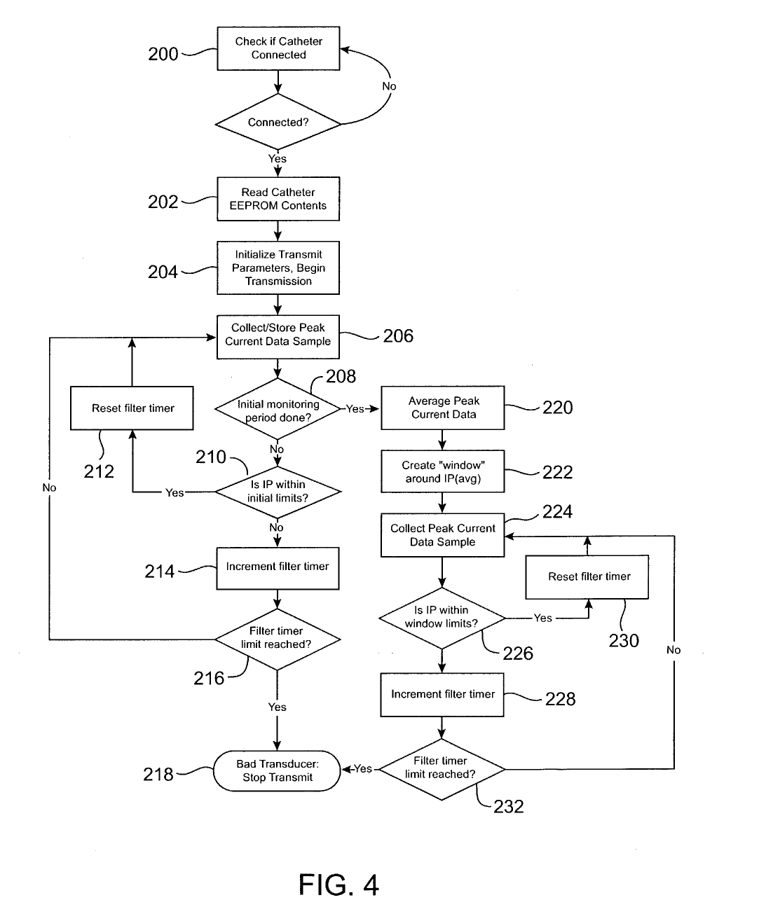

In accordance with the present invention, the software operating on computer 105 executes an algorithm to determine the condition of transducers 150, 160 and 170 during operation, as detailed by the flowchart of FIG. 4. Specifically, upon connection of catheter 180 to system 100, computer 105 reads information from the EPROM device 120 located in catheter 180. This information preferably includes information about the catheter type and configuration, transducer characteristics including resonant frequency and efficiency, and operating characteristics including peak current levels measured during manufacturing test (i.e., test current, I_{T}). Analysis to determine if transducer failure has occurred is preferably carried out as follows, in accordance with the preferred method described herein.

At step 200, a determination is made as to whether the catheter is connected. At step 202, the data in memory device 120 are read by computer 105. At step 204, the computer 105 configures frequency generator and timing circuitry 125 and power amplifier 130 to produce the appropriate excitation waveforms for the catheter, and catheter operation is commenced. At step 206, measurement of I_{P} is started. Such measurement is carried out over time period T1 (step 208). Determination is made continually during time period T1 (at step 210) whether I_{P} has moved out of range 20.

In various optional preferred aspects, temporal filtering is implemented by steps 210, 212, 214, and 216 to allow brief excursions of I_{P} out of range 20. If I_{P} has exited range 20, a determination is made at step 216 whether such deviation was for at least as long as time period T3. If so, a transducer failure conclusion is made at step 218. This is advantageous, because as described previously, certain events such as the injection of x-ray contrast media into the artery can cause significant changes in the acoustic environment that temporarily affects the impedance, and thus the peak current, of the transducer. Accordingly, if I_{P} remains beyond desired limits longer than the specified time T3, computer 105 can then determine that one or more of the transducers are defective and appropriate action may then be taken, for example shutting off the excitation signal to the transducer and notifying the operator of the failure.

After the passage of time period T1, I_{AVG} is calculated from the I_{P} measurements obtained during T1 at step 220. Thereafter, at step 222, range 30 is determined, and at step 224, I_{P} is measured during time period T2. At step 226, a determination is made whether I_{P} has exited range 30.

Again, temporal filtering is incorporated to allow brief excursions of I_{P} out of range 30. This filtering is implemented by steps 226, 228, 230, and 232. If I_{P} has exited range 30, a determination is made at step 232 whether such deviation was for at least as long as time period T3. If so, a transducer failure conclusion is made at step 218 and appropriate action may be taken.

In accordance with a second aspect of the present method, transducer failure can also be detected as follows. Referring to FIG. 5, which illustrates instantaneous transducer current I_{TR} during a single burst (such as B1 or B2 in FIG. 2) and the detected peak current I_{P} for a properly functioning transducer, it can be seen that transducer current I_{TR} reaches a peak at about the second cycle 43 of the waveform, and then settles down to a steady level at and beyond the fifth cycle 44. As can be seen, the amplitude of second cycle peak 43 is normally about 25-30% higher than the steady-state current at or beyond the fifth cycle 44.

This feature of the properly operating transducer's waveform is not seen in FIG. 6 (which illustrates the current waveforms I_{TR} and I_{P} for a degraded transducer) in which little or no peaking occurs (i.e., the transducer current amplitude remains approximately constant from near the second cycle peak throughout the transmit waveform).

In accordance with an optional aspect of the present invention, the amplitude of second cycle peak 43 (of I_{TR}) is thus compared to the amplitude of the fifth cycle peak 44 to determine whether or not the transducer is functioning properly, or has degraded.

Specifically, should the value of fifth cycle peak 44 rise above a predetermined percentage, for example 65% to 75% of the value of second cycle peak 43, the present system concludes that transducer failure has occurred.

Referring to FIG. 3B, an optional system for accomplishing this optional preferred aspect is provided as follows. A second peak detector 102 is incorporated into system 100. Preferably, second detector 102 has a response time such that the value of the maximum (i.e., second cycle peak 43) is held and digitized. Software in computer 105 then compares the digitized values of the second cycle peak 43 and the fifth cycle peak 44 from detectors 102 and 101.

Preferably, both absolute minimum and maximum values for each of current cycle peaks 43 and 44 (and allowable ratios between cycle peaks 43 and 44) could be stored in memory device 120 in catheter 180. An advantage of pre-storing these values in the particular memory device 120 for each individual catheter 180 is that the present system is able to accommodate catheters having different characteristics, each having unique pre-stored values. Since comparisons between measured values and pre-stored values are made with the software in computer 105, the present invention advantageously compensates for various transducer configurations and manufacturing variances.

An advantage of using this second aspect of the preferred method for transducer failure detection is that the requirement of separating transducer operation and monitoring into two time periods T1 and T2 (referring to FIG. 1) may not be required. This is because in-vivo conditions causing transducer current I_{TR} to vary from test current I_{T} affect the entire waveform equally and the characteristic peaking of a working transducer would not be lost. A larger peak current operating range (such as range 20) could be maintained for detection of non-transducer related failures (e.g., electrical short or open circuits) while the ratio of cycle peaks 43 and 44 would be used to detect degraded transducer element(s). This could eliminate the need to rely on the large operating range 20 for the detection of transducer degradation during the time period TI thus improving sensitivity during the initial operating period, as well as allowing simplification of the monitoring algorithm.

Another advantage of using this second aspect of the preferred method for transducer failure detection is that a “transient filter” (i.e., allowable I_{P} excursions out of range 20 for less than time period T3) as described above, may not be required. Alternatively, if such a transient filter is used, T3 could be of a shorter length, due to the fact that if current changes due to a temporary change in acoustic load, the entire waveform is changed equally and the characteristic peaking (at cycle peak 43) of a working transducer would not be lost. This could allow for more rapid software detection of a degraded transducer.

Referring to FIG. 3C, another optional system for accomplishing this optional preferred aspect is provided wherein circuitry performs a simple comparison between second cycle peak 43 and fifth cycle peak 44, and provides a suitable output when fifth cycle peak 44 reaches a set percentage of the second cycle peak 43. Specifically, a comparator 103 can be used to compare the peak signals received from second cycle peak current detector 102 and the fifth cycle peak current detector 101. Comparator 103 could operate independently of the software to shut down transducer operation, if transducer failure is detected. As such, the present system need not require computer functions dedicated to monitoring transducer function. Alternatively, such a system could be used as a backup that would still operate in the event of a computer failure.

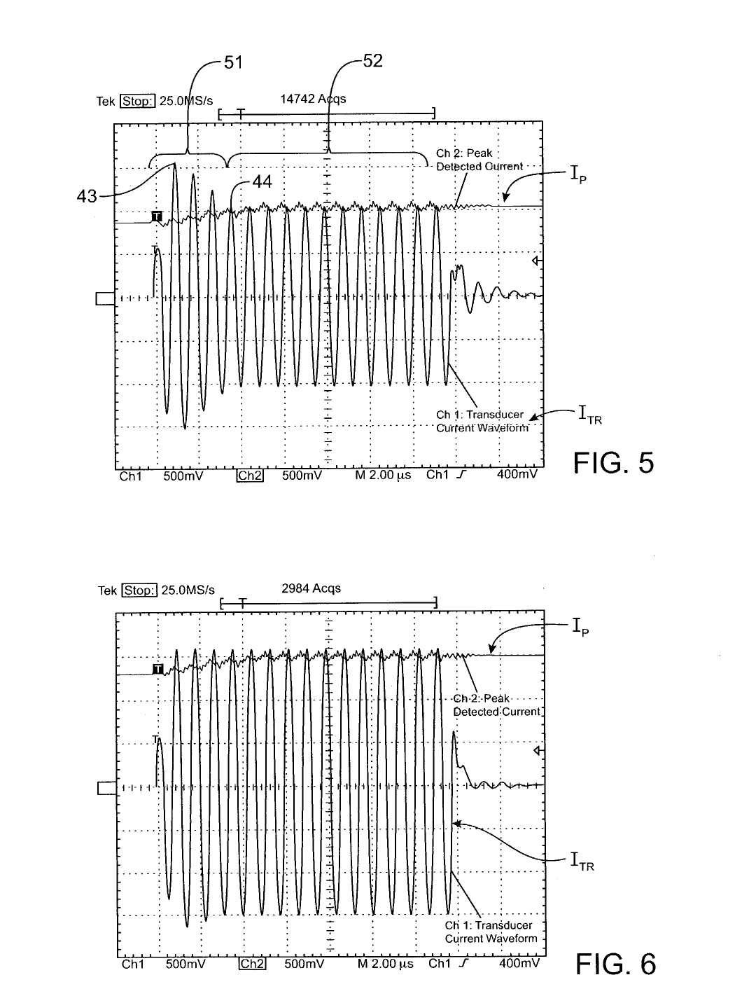

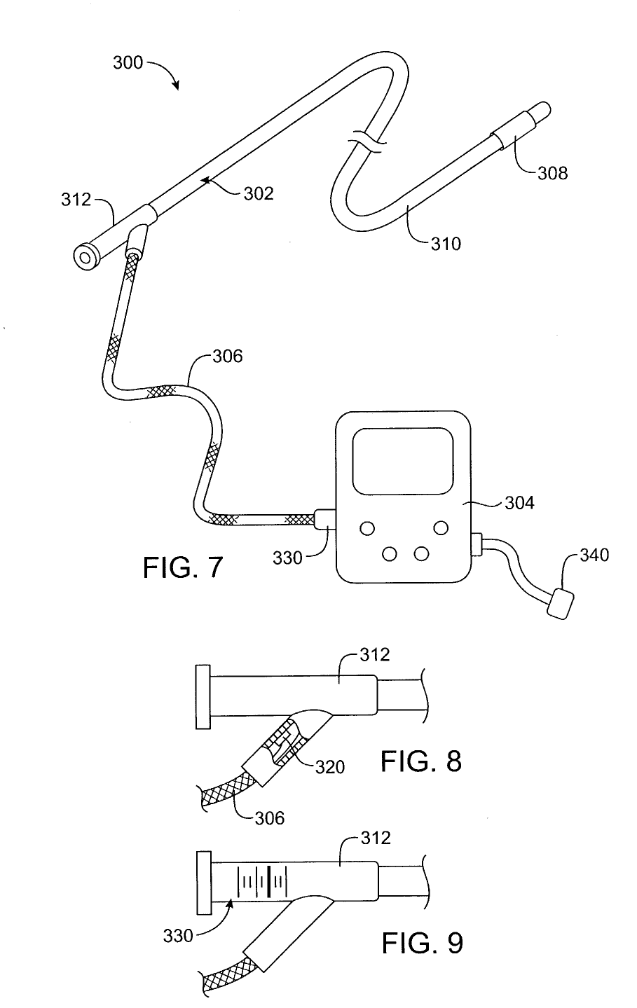

Referring now to FIG. 7, a system 300 constructed in accordance with the principles of the present invention comprises a high-output therapeutic ultrasound catheter 302 connected to a therapeutic ultrasound controller 304 by a cable 306. The high-output therapeutic ultrasound catheter 302 includes an ultrasound transducer assembly 308 at or near the distal end of catheter body 310. A hub assembly 312 is located on the proximal end of the catheter body 310. The catheter 302 is typically an intravascular catheter intended for treatment of the coronary arteries or other portions of a patient's vasculature. Specific examples of high-output therapeutic intravascular catheters are provided in the patents and pending applications of the assignee of the present application, as listed and incorporated by reference hereinabove.

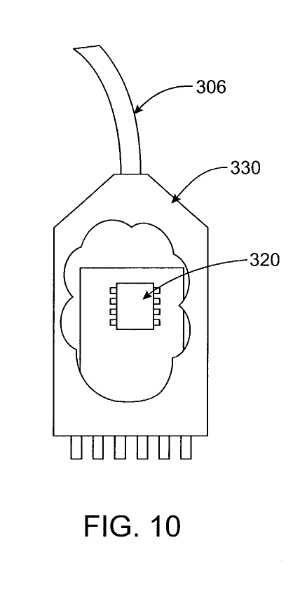

The catheter 302 is provided with data representing a measured operational range of the transducer of assembly 308. The data will be stored or otherwise made available on the catheter 302, i.e., either on or in the catheter body 310, the transducer assembly 308, in or on the hub 312, or most usually in or on the catheter electrical connector 330 that plugs into the controller 304. Referring to FIG. 10, the most common way for incorporating the ultrasound data with the system 300 is by providing an electronic memory module 320, shown mounted within the electrical connector 330. The memory module 320 can take a variety of conventional forms, typically being a flash memory or EEPROM which will retain the data even in a non-powered configuration. The electronic memory module may be connected to the controller through or by conventional wiring or other data transmission elements within the cable 306. In addition to conventional wiring, it would be possible to provide optical connections between the memory module 320 and the controller 304. The data in the memory module 320 represents the measured operational range of the ultrasonic transducer, either in the form of an average or peak operational value, the calculated operational range, or any other data upon which the expected operational range may be determined or read by the controller 304. The memory module could alternatively be located in the hub 312, as shown in FIG. 8.

As an alternative to the electronic memory module 320, the hub 312 or electrical connector 330 may be provided with printed, embossed, or otherwise physically marked indicia, typically in the form of a barcode 330, as shown in FIG. 9. The barcode 330 may incorporate the same information as stored in the memory module 320, but will typically be read by an external scanner 340 which may optically be connected to the controller 304. In additional to such machine readable data, the hub, catheter body, or electrical connector body may also incorporate human readable data, typically simple number or alphanumeric patterns, which may then be transferred by the end user of the controller using a keyboard or other interface. A variety of other approaches for incorporating the operational data of the transducer into or onto the catheter will also be available, although it is generally believed that the electronic memory provides the most efficient approach since the controller may automatically read the data from the electronic memory at the beginning of any treatment protocol.

The above-described examples of method and associated apparatus can advantageously be extended to function with a variety of single- and multiple-transducer and catheter configurations to allow rapid and reliable detection of transducer failure during operation. Detection of such failure provides a necessary level of patient safety by terminating ultrasound transmission in the event of transducer failure before excessive heating can occur.

What is claimed is:

1. A method for detecting failure of an ultrasound transducer in a remotely positioned therapeutic device, said method comprising:
determining a first operational range for the transducer based on data stored in or on the device; and
observing operation of the transducer over a first time period, wherein a failure is detected if the transducer operates outside the determined first operational range.

2. A method as in claim 1, further comprising:
determining a second operational range for the transducer based on the observed operation during the first time period, wherein the second operational range is narrower than the first;
observing operation of the transducer during a second time period extending after the first time period, wherein a failure is detected if the transducer operates outside of the determined second operational range.

3. A method as in claim 1 or 2, wherein determining the first operational range comprises calculating a range based on a value provided by the data stored on or in the device.

4. A method as in claim 3, wherein the value is a test current and the calculated range is above the test current value.

5. A method as in claim 4, wherein the first operational range is a current from 110% to 130% of the test current value.

6. A method as in claim 1 or 2, wherein the first time period is from five seconds to thirty seconds measured from the time the transducer is initially energized.

7. A method as in claim 6, wherein the failure of the transducer during the first operational period is detected when the transducer operates outside the determined first operational range for a minimum time period.

8. A method as in claim 7, wherein the minimum time period is fifteen seconds.

9. A method as in claim 1 or 2, wherein observing operation of the transducer comprises measuring the peak current value of a plurality of sequential excitation bursts and calculating the peak current value after at least four current waveform cycles of each excitation burst.

10. The method of claim 9, wherein the peak current is calculated after at least five current waveform cycles of each excitation burst.

11. A method as in claim 2, wherein determining the second operational range comprises calculating an average power consumption value of the transducer during the first operational range, wherein the second operational range is from 95% to 105% of the calculated average power consumption during the first period.

12. A method as in claim 2, wherein the failure is detected only if the transducer operates outside of the determined second operational range for a minimum time period.

13. A method as in claim 12, wherein the minimum time period is 15 seconds.

14. A method for detecting failure of an ultrasound transducer in a remotely positioned therapeutic device, said method comprising:
measuring peak current resulting from individual cycles of the excitation voltage to the transducer during operation; and
calculating the difference between peak current of an early cycle with peak current of a later cycle, wherein a calculated difference below an expected minimum value indicates transducer failure.

15. A method as in claim 14, wherein the difference between the second cycle peak current and the fifth or subsequent cycle peak current is calculated.

16. A method as in claim 15, wherein the expected difference is at least 25% of the second cycle peak current.

17. A therapeutic ultrasound controller for use in combination with a catheter having a high-output therapeutic ultrasound transducer, said controller comprising:
means for measuring peak current delivered to the transducer;
means for determining a first expected peak current operational range for the transducer; and
means for comprising the measured peak current value with the first determined peak current range, wherein a measured peak current value which falls outside of the first determined peak current range indicates a transducer failure.

18. A therapeutic ultrasound controller as in claim 17, wherein the determining means receives a value from an electronic memory in the catheter.

19. A therapeutic ultrasound controller as in claim 17 or 18, further comprising:
means for determining a second expected peak current operational range based on the peak current measured during a first operational period; and
means for comprising the measured peak current value with the second determined peak current range, wherein a measured peak current value which falls outside of the second peak current grange indicates a transducer failure.

20. A therapeutic ultrasound controller for use in combination with a catheter having a high-output therapeutic ultrasound transducer, said controller comprising:
means for measuring peak current to the transducer; and
means for comparing the measured peak current of an early cycle with the measured peak current of a later cycle.

21. A therapeutic ultrasound controller as in claim 20, wherein the comparing means compares the peak current of a second cycle in a burst with the peak current of a fifth or later cycle in the same burst, wherein a difference of less than 25% of the second cycle peak current indicates a failure of the transducer.

22. An intravascular catheter comprising:
a catheter body;
a high-output therapeutic ultrasound transducer operatively disposed on the catheter body; and
data on the catheter body representing a measured operational range of the transducer.

23. An intravascular catheter as in claim 22, wherein the high-output therapeutic ultrasound transducer has a power output of at least 100 watts.

24. An intravascular catheter as in claim 22, further comprising an electronic memory device, wherein the data are stored in the device.

25. An intravascular catheter as in claim 24, wherein the electronic memory device is selected from the group consisting of flash memory, RFID's, and EEPROM's.

26. An intravascular catheter as in claim 25, wherein the data comprises indicia printed on the catheter body.

27. An intravascular catheter as in claim 22, wherein the printed data comprises machine readable code.

28. An intravascular catheter as in claim 22, wherein the printed data comprises human readable information.

29. A method for fabricating an intravascular catheter, said method comprising:
providing an intravascular catheter having a catheter body and a high-output therapeutic ultrasound transducer operatively disposed on the catheter body;
measuring a power consumption characteristic of the transducer during operation; and
embedding the data in or on the catheter body in a readable form.

30. A method as in claim 29, wherein measuring comprises measuring current consumption.

31. A method as in claim 30, wherein current is measured with the transducer immersed in water and under nominal excitation parameters.

32. A method as in claim 29, wherein embedding comprises storing the data in an electronic memory disposed in or on the catheter body.

33. A method as in claim 29, wherein embedding comprises printing indicia setting forth the measured value on the current catheter body.

34. A method as in claim 33, wherein the indicia are machine readable.

35. A method as in claim 33, wherein the indicia are human readable.
